(19) United States
(12) Patent Application Publication
(10) Pub. No.: US 2025/0003909 A1
(43) Pub. Date: Jan. 2, 2025

(54) PORTABLE AIR SAMPLING SYSTEM FOR DETECTING VOLATILE ORGANIC COMPOUNDS

(71) Applicant: The Research Foundation for the State University of New York, Albany, NY (US)

(72) Inventors: Chuan-Jian Zhong, Vestal, NY (US); Shuxia Lu, Vestal, NY (US); Guojun Shang, Vestal, NY (US); Shan Yan, Medford, NY (US); Jin Luo, Vestal, NY (US); Dong Dinh, Rochester, MN (US)

(21) Appl. No.: 18/625,659

(22) Filed: Apr. 3, 2024

Related U.S. Application Data
(60) Provisional application No. 63/456,961, filed on Apr. 4, 2023.

(51) Int. Cl. G01N 27/12; G01N 33/497

(52) U.S. Cl. CPC G01N 27/12; G01N 33/497

(57) ABSTRACT

Air sampling sensor and system for detecting volatile organic compounds and applications thereof. A nanoparticle-structured chemiresistive sensor array in the analyzer generates an output signal based on the presence of selected volatile organic compounds in an air sample. The sensors of the array include sensing films from the group of molecularly-linked gold nanoparticles with different linking and capping molecules and gold nanoparticles of different sizes. A micro-processing unit analyzes the output signal of the sensor array and indicates the presence or absence of selected volatile organic compounds. The air sampling system can be used to analyze human breath and report the presence of volatile organic compounds relevant to lung cancer biomarkers. The air sampling system can also be used as an environmental air monitoring system to detect the presence of hazardous chemicals in the air.

### CROSS-REFERENCE TO RELATED APPLICATIONS

This application claims the benefit of U.S. Provisional Patent Application No. 63/456,961, filed Apr. 4, 2023, the entirety of which is hereby incorporated herein by this reference.

### STATEMENT REGARDING FEDERALLY SPONSORED RESEARCH

This invention was made with government support under IIP 1640669 awarded by the National Science Foundation. The government has certain rights in the invention.

### BACKGROUND OF THE INVENTION

1. Field of the Invention

The present invention generally relates to airborne chemical detection systems. More particularly, the present invention relates to a portable air sampling system for detecting volatile organic compounds.

2. Description of the Related Art

A chemiresistor is a material that changes electrical resistance in response to changes in the nearby chemical environment and, therefore, can be used as chemical sensors. Chemiresistors typically rely on a direct chemical interaction between the sensing material and an analyte, where the sensing material and the analyte interact by covalent bonding, hydrogen bonding, or molecular recognition. There are several different known materials which have chemiresistor properties, such as metal-oxide semiconductors, conductive polymers, and nanomaterials such as graphene, carbon nanotubes and nanoparticles. Chemiresistors are often used as partially selective sensors in devices like “electronic tongues” or “electronic noses.”

A basic chemiresistor typically consists of a sensing material that bridges the gap between two electrodes, or can be a coating a set of interdigitated electrodes, and the resistance between the electrodes is then measured. The sensing material has an inherent resistance that can be modulated by the presence or absence of an analyte. During exposure, the analyte interacts with the sensing material to cause changes in the resistance reading. In some configurations of chemiresistors, resistance changes simply indicate the presence of analyte, while in others, resistance changes are proportional to the amount of analyte present. In this configuration, the chemiresistor allows for the amount of analyte present to be measured. Consequently, chemiresistor sensing is often used in environmental and healthcare applications for detection of chemicals and compounds in an air sampling.

However, in healthcare applications, existing sensor systems that use chemiresistors require large electronic interfacing circuits, and thus are not suitable for portable devices in field or personalized monitoring or screening applications. These systems also have durability and reliability issues that must be overcome for practical applications and repeated economic usage. One type of chemiresistor used in healthcare is as a tool for early-stage cancer detection from sensors that detect breath volatile organic compounds (VOCs) as biomarkers in exhaled human breaths. However, a major challenge to this type of system is the lack of effective integration of the different sensor system components toward the desired portability, sensitivity, selectivity, and durability for many of the current breath sensors.

The present invention therefore addresses the problems associated with the use of chemiresistors to detect chemicals and compounds, particularly in medical diagnoses and testing.

### BRIEF SUMMARY OF THE INVENTION

Briefly described, the present invention provides tools, devices, systems, and methods for early-stage cancer detection utilizing air sampling sensors according to the present invention that detect volatile organic compounds (VOCs) as biomarkers in exhaled breaths or VOCs in the air. In embodiments, the present invention provides a portable, wireless breath sensor testing system integrated with sensor electronics, breath sampling, data processing, and sensor arrays derived from nanoparticle-structured chemiresistive sensing interfaces for detection of VOCs relevant to lung cancer biomarkers in human breaths. The sensor arrays exhibit high sensitivity to lung cancer VOC biomarkers and mixtures. In embodiments, the air sampling sensors are also used for environmental air monitoring to detect hazardous chemicals and VOCs.

In one embodiment, the invention includes a portable and wireless breath sensor testing system integrated with sensor electronics, breath sampling, data processing, and sensor arrays derived from nanoparticle-structured chemiresistive sensing interfaces for detection of VOCs relevant to lung cancer biomarkers in human breaths. In addition to showing the sensor viability for the targeted application by theoretical simulations of chemiresistive sensor array responses to the simulated VOCs in human breaths, the sensor system was tested experimentally with different combinations of VOCs and human breath samples spiked with lung cancer-specific VOCs. The sensor array exhibits high sensitivity to lung cancer VOC biomarkers and mixtures, with a limit of detection as low as 6 ppb. The results from testing the sensor array system in detecting breath samples with simulated lung cancer VOC constituents have demonstrated an excellent recognition rate in discriminating healthy human breath samples and those with lung cancer VOCs.

In one embodiment, an air sampling system for detecting volatile organic compounds according to the present invention includes a chemiresistive sensor array, a multichannel sensor array interface board electrically coupled to the chemiresistive sensor array, an analog-to-digital converter electrically coupled to the multichannel sensor array interface board, and a micro-processing unit electrically coupled to the analog-to-digital converter. The chemiresistive sensor array produces an electrical signal whenever a volatile organic compound is present in an air sample introduced into a chamber housing the chemiresistive sensor array.

In an embodiment, channels of the multichannel sensor array interface board are configured to measure a resistance in the range of 30 ohms to 30 megaohms, and the channels automatically adjust to particular resistance measurement ranges.

In an embodiment, the chemiresistive sensor array of the air sampling sensor includes sensing films from the group of molecularly-linked gold nanoparticles of different sizes, e.g., MUA-Au₂ₙₘ, MUA-Au₅ₙₘ, BDT-Au₂ₙₘ, BDT-Au₅ₙₘ, PDT-Au₂ₙₘ, HDT-Au₂ₙₘ, NDT-Au₂ₙₘ, and ND-Au₅ₙₘ, where MUA represents mercaptoundecanoic acid, BDT butanedithiol, PDT pentanedithiol, HDT hexadecanedithiol, NDT nonanedithiol, Au₂ₙₘ gold nanoparticles of 2 nm diameter, and Au₅ₙₘ gold nanoparticles of 5 nm diameter, and the micro-processing unit performs principal component analysis of data related to electrical signals produced by the chemiresistive sensor array to determine the presence of volatile organic compounds in the air sample.

In an embodiment, the chemiresistive sensor array comprises sensing films selected to detect the presence of, for examples, toluene, styrene, o-xylene, dimethyl ether, 1,3-pentadiene, methyl hydrazine, ethanol, ethyl benzene, and/or 2-methly-hexane in the air sample.

In an embodiment, the chemiresistive sensor array comprises sensing films selected to detect the presence of, for examples, 2-ethyl-1-hexanol, 2-ethyl-4-methyl-1-pentanol, 2,3,4-trimethyl-pentant, and/or 4-methyl-octane in the air sample.

In embodiments, the chemiresistive sensor array includes sensing films selected to detect the presence of volatile organic compounds in the air sample associated with lung cancer.

In an embodiment, the chemiresistive sensor array comprises sensing films selected to detect the presence of volatile organic compounds in the air sample not associated with lung cancer.

The present invention therefore provides an advantage in providing a portable chemical sensing system that detects volatile organic chemicals in an air sample. The present invention further has industrial application in the creation of medical devices that can detect compounds, such as VOCs, in human breath for purposes of medical diagnosis. This and other advantages and applications of the present invention will be apparent to one of skill in the art.

### BRIEF DESCRIPTION OF THE DRAWINGS

FIG. 1 illustrates an example portable air sampling system for detecting volatile organic compounds according to an embodiment of the present invention.

FIG. 2 illustrates an example portable air sampling sensor according to an embodiment of the present invention.

FIG. 3 illustrates the portable air sampling sensor of FIG. 2 communicating wirelessly with a computer.

FIG. 4 illustrates a human providing a breath sample to be tested using a portable air sampling system for detecting volatile organic compounds according to an embodiment of the present invention.

FIG. 5 illustrates the portable air sampling sensor of FIG. 2 being used to analyze a human breath sample for detecting volatile organic compounds according to an embodiment of the present invention.

FIG. 6 illustrates the electronic circuitry of a portable air sampling sensor for detecting volatile organic compounds according to an embodiment of the present invention.

FIG. 7 illustrates an example embodiment of an eight-channel chemiresistor array sensor for detecting volatile organic compounds according to the present invention.

FIG. 8 illustrates an embodiment of an example chemiresistor sensor for detecting volatile organic compounds according to the present invention.

FIG. 9 illustrates an example output of a chemiresistor sensor for detecting volatile organic compounds according to an embodiment of the present invention.

FIG. 10 illustrates an example output of an eight-channel chemiresistor array sensor for detecting volatile organic compounds according to an embodiment of the present invention.

FIG. 11 illustrates various statistical methods used to process sensor data according to embodiments of the present invention.

FIG. 12 illustrates various principal component analysis plots of sensor array responses according to embodiments of the present invention.

FIG. 13 illustrates example sensor responses of a sensor array according to an embodiment of the present invention to human breath samples.

FIG. 14 illustrates a principal component analysis plots of sensor array responses according to embodiments of the present invention.

FIG. 15 illustrates a further principal component analysis plots of sensor array responses according to embodiments of the present invention.

FIG. 16A illustrates an example graphical user interface for a portable air sampling system for detecting volatile organic compounds.

FIG. 16B illustrates a further example of the graphical user interface of FIG. 16A.

FIG. 16C illustrates a further example of the graphical user interface of FIGS. 16A-16B.

FIG. 16D illustrates a further example of the graphical user interface of FIGS. 16A-16C.

FIG. 16E illustrates a further example of the graphical user interface of FIGS. 16A-16D.

FIG. 17 illustrates an example handheld air sampling sensor for detecting volatile organic compounds according to an embodiment of the present invention.

FIG. 18 illustrates an air monitoring system for remotely monitoring a large number of air sampling sensors for detecting volatile organic compounds according to an embodiment of the present invention.

### DETAILED DESCRIPTION OF THE INVENTION

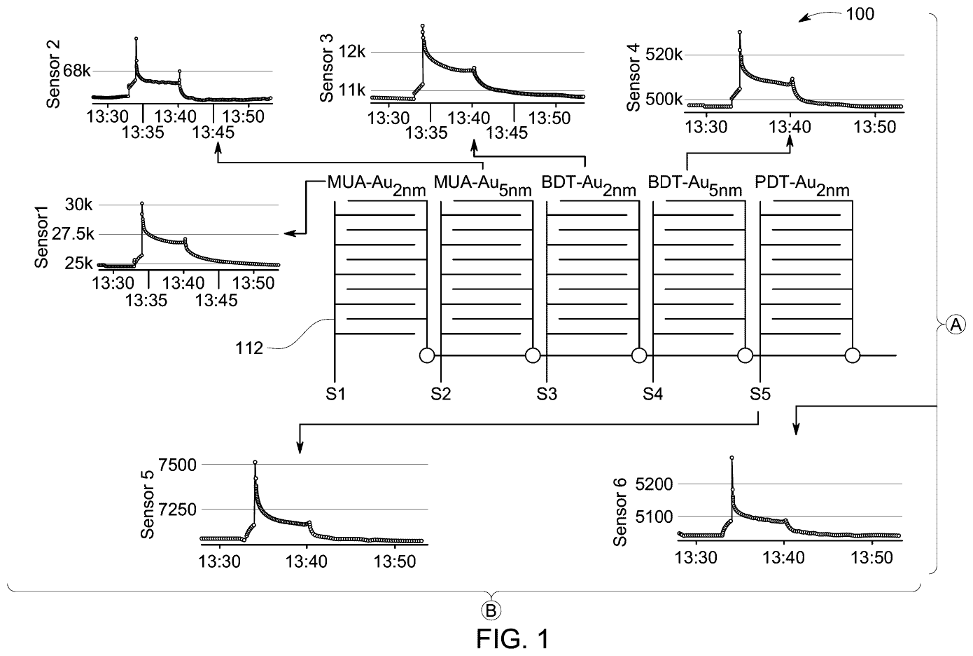

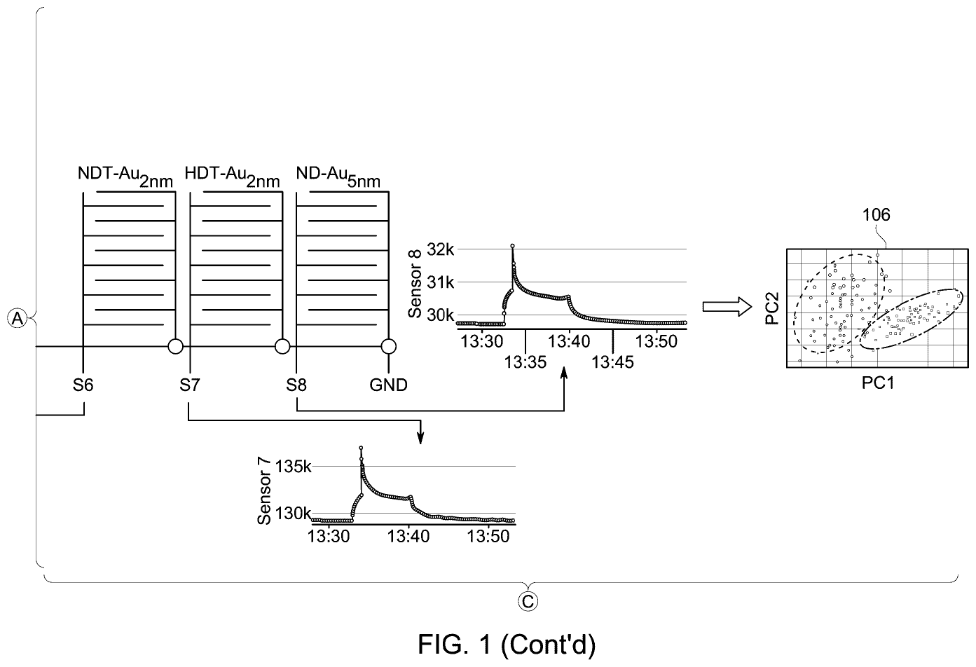

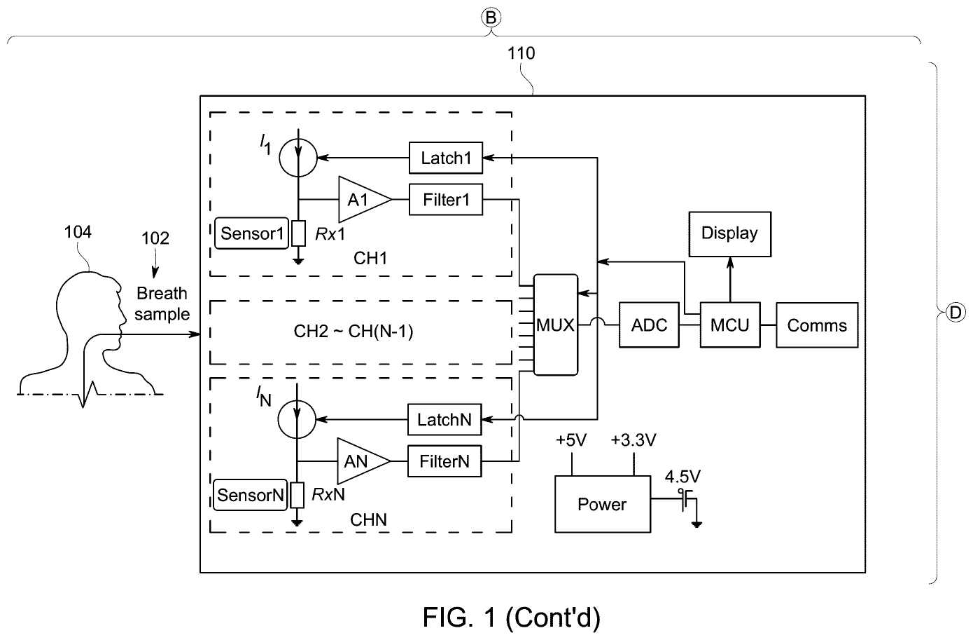

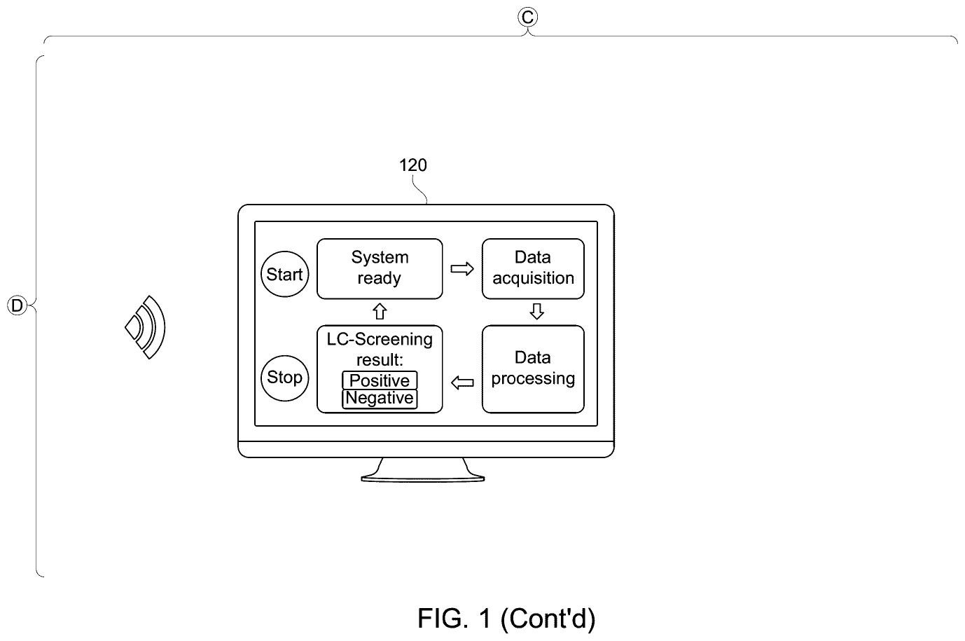

With reference to the figures in which like numerals represent like elements throughout the several views, FIG. 1 is a diagrammatic view of the present inventive system 100. The timely screening of lung cancer is important for early diagnosis and treatment, which requires reliable, low-cost, and noninvasive detection tools. Presented herein are tools, for example, devices, systems, and methods for early-stage cancer detection utilizing breath analyzers and/or sensors that detect breath volatile organic compounds (VOCs) as biomarkers in exhaled breaths. In embodiments, the present invention provides a portable, wireless breath sensor testing system integrated with sensor electronics, breath sampling, data processing, and sensor arrays derived from nanoparticle-structured chemiresistive sensing interfaces for detection of VOCs relevant to lung cancer biomarkers in human breaths. The sensor arrays described herein exhibit high sensitivity to lung cancer VOC biomarkers and mixtures, with a limit of detection in embodiments as low as 6 ppb. In embodiments, the analyzers and sensors described herein are also used for environmental air monitoring to detect hazardous chemicals.

FIG. 1 illustrates an example portable air sampling system 100 for detecting volatile organic compounds according to an embodiment of the present invention. As depicted in FIG. 1, in embodiments a human breath sample 102 is provided by a human 104 to a portable air sampling sensor 110 or air sampling analyzer 110. The portable air sampling sensor 110 includes a multichannel chemiresistor array sensor 112 that detects the presence of VOCs in the breath sample 102. Each of the sensors of the multichannel chemiresistor array sensor 112 generates an output representative of the different concentrations of the VOCs present in breath sample 102. As explained in more detail below, the outputs of each of the sensors are analyzed using statistical methods in order to determine which VOCs are present in the breath sample 102 as shown in graph 106. In embodiments, the detection of VOCs relevant to lung cancer biomarkers in the human breath 102 is wirelessly transmitted to a computer and displayed on a display 120 as illustrated in FIG. 1.

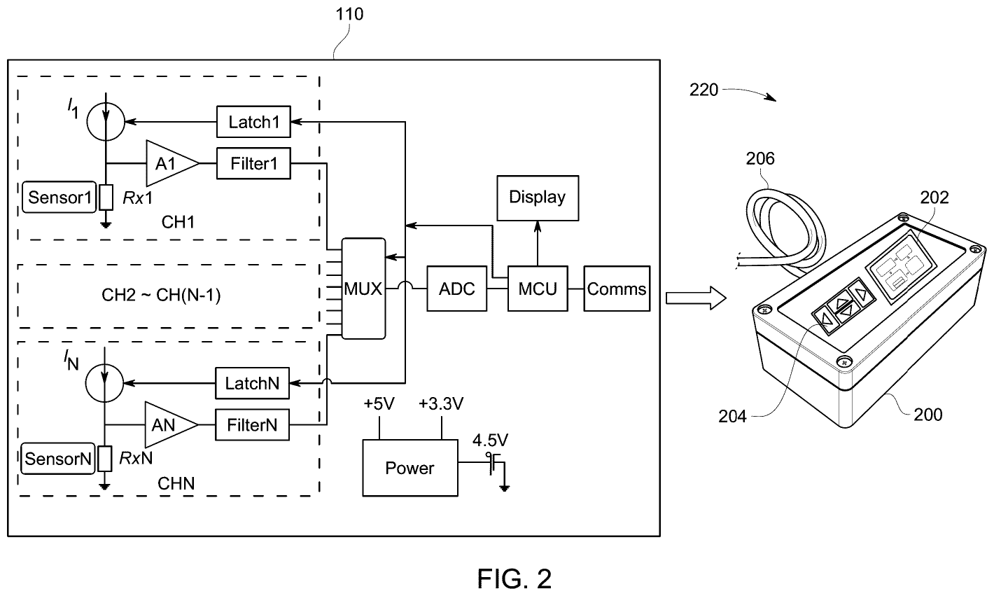

FIG. 2 further illustrates portable air sampling sensor 110 according to an embodiment of the present invention. As shown in FIG. 2, in embodiments portable air sampling sensor 110 is housed in an enclosure 200. Enclosure 200 includes a local user display 202 and control buttons 204. A flexible hose 206 is used for introducing air samples to be analyzed for the presence of VOCs by portable air sampling sensor 110. Portable air sampling sensor 110 together with an enclosure and other components described herein used to analyze an air sample are referred to as a portable air sampling sensor 220.

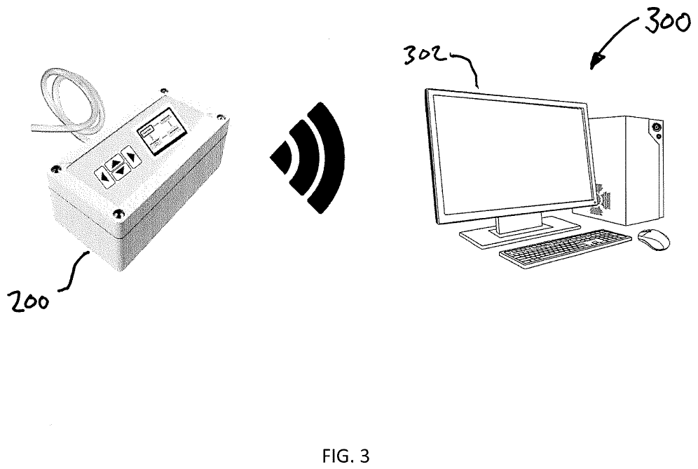

FIG. 3 illustrates that in embodiments portable air sampling sensor 110 can transmit data wirelessly to a computer 300. Computer 300 may be for example a computer in a doctor's office or a cloud server computer. Computer 300 makes it easier to view data generated by portable air sampling sensor 110 for example because of the larger display 302 coupled to computer 300. Computer 300 may also be used to gather data from more than one portable air sampling sensor 110 for storage and subsequent viewing and analysis.

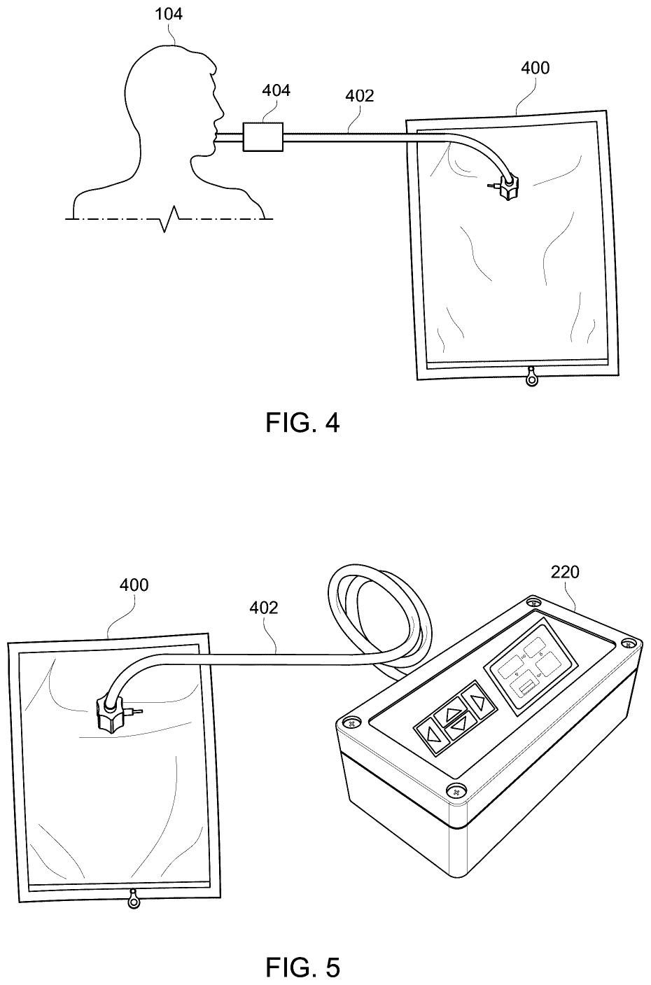

FIG. 4 illustrates a human 104 providing a breath sample to be tested using a portable air sampling system 100 for detecting volatile organic compounds according to an embodiment of the present invention. As shown in FIG. 4, human 104 blows air into a collection bag 400 using a tube 402. In embodiments, tube 402 includes a filter 404. The purpose of filter 404 for example is to remove moisture from the breath sample being provided by human 104.

FIG. 5 illustrates the introduction of a breath sample contained in collection bag 400 being introduced into portable air sampling sensor 220. After the breath sample is introduced into portable air sampling sensor 220, portable air sampling sensor 220 analyzes the human breath sample for the presence of VOCs as described in more detail below. Tube 402 connects the collection bag 400 to the portable air sampling sensor 220 so that the breath sample can be transferred from collection bag 400 to portable air sampling sensor 220.

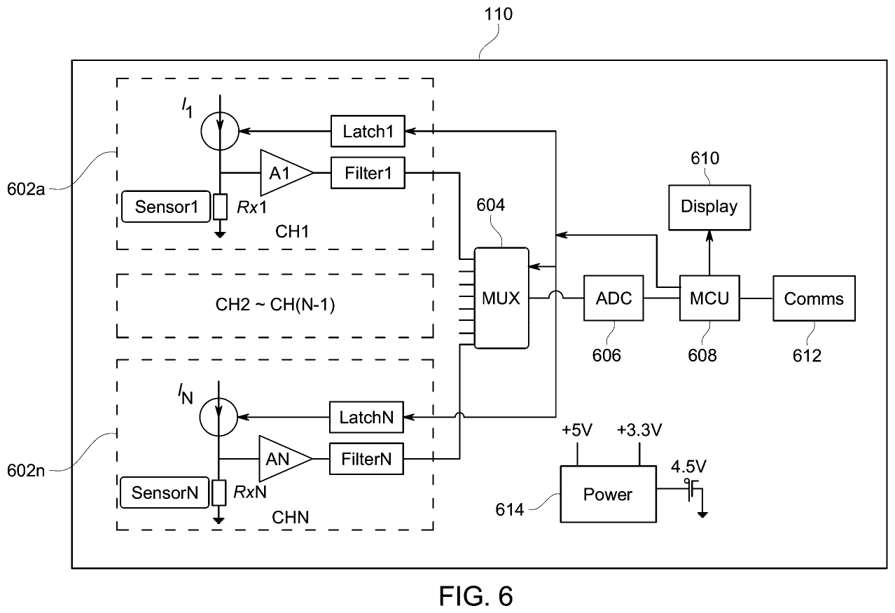

FIG. 6 further illustrates the electronic circuitry of a portable air sampling sensor for detecting volatile organic compounds according to an embodiment of the present invention such as portable air sampling sensor 110 or portable air sampling sensor 220. As shown in FIG. 6, a portable air sampling sensor 110 includes multiple sensor channels 602a through 602n. The output of each sensor channel is an analog signal. A multiplexer (MUX) 604 is used to sample the outputs of the various sensor channels 602 and pass the sampled outputs to an analog-to-digital converter (ADC) 606. The digital signal generated by analog-to-digital converter 606 can be read by and processed by micro-processing unit (MCU) 608, as described in more detail below. In embodiments, the results of the data processing performed by micro-processing unit 608 are presented to a user on display 610. The results may also be transmitted to another device such as a computer 300 using communications module 612. In embodiments, this communications module can implement any known communications protocol. In embodiments, communications module 612 implements a wireless communications protocol that can communicate with other computers for example using the Internet.

In embodiments, the portable air sampling sensor 110 or portable air sampling sensor 220 is designed for rapid measurement of the resistance changes of the nanostructured chemiresistor array. The resistance of different sensors in the array may vary greatly, for example spanning several orders of magnitude. Based on the sensors' resistance ranges, the measured resistance is designed to cover a wide range (e.g., 30 Ω˜300 MQ). In one embodiment, the nanostructured chemiresistor array consists of 8 sensor channels. In other embodiments, it can be expanded to more than eight sensor channels such as for example 16, 24, or 32 channels as desired. In an embodiment, in order to improve the measurement accuracy of the resistance change ratio (ΔR/Ri), the measurement range of each channel is subdivided into 16 ranges. The current used for measuring the resistance change is as low as a few nA. See A Low-Current and Multi-Channel Chemiresistor Array Sensor Device, Wang Z, Shang G, Dinh D, Yan S, Luo J, Huang A, Yang L, Lu S, Zhong CJ, Sensors (Basel), 2022 Apr. 5;22 (7): 2781. doi: 10.3390/s22072781. PMID: 35408393; PMCID: PMC9003399, which is incorporated herein by reference in its entirety. The output is the resistance values of the sensors, which are displayed for example in two ways: (1) by a display on the portable air sampling sensor, and (2) by a computer using communications module 612.

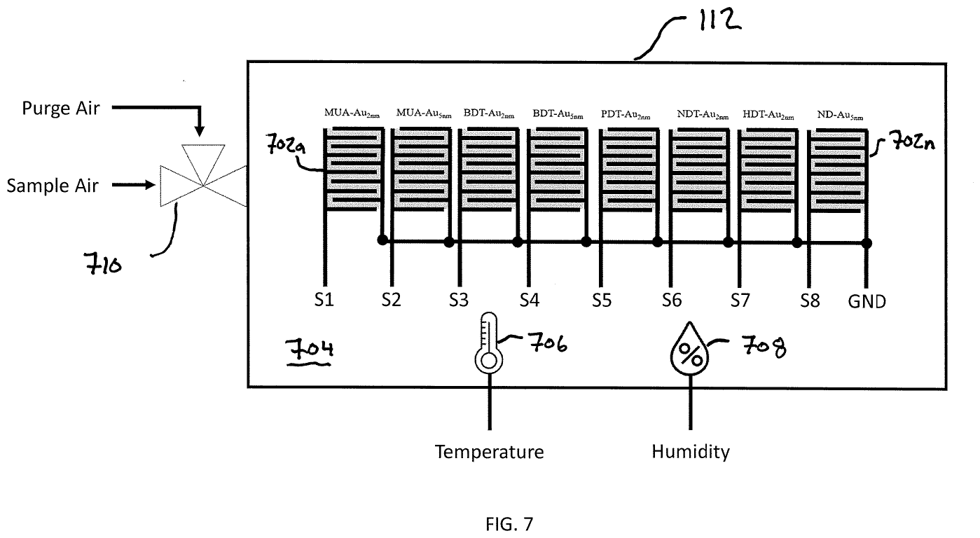

FIG. 7 illustrates an example embodiment of a multichannel chemiresistor array sensor 112. In an embodiment, multichannel chemiresistor array sensor 112 has eight sensor channels for detecting volatile organic compounds according to the present invention. In other embodiments, multichannel chemiresistor array sensor 112 has more or less than eight sensor channels. In such embodiments, multiple air samples can be tested at once in the multichannel array sensor 12, or the same sample can be tested simultaneously for the presence of various VOCs.

A “chemiresistor” is a material that changes its electrical resistance in response to changes in its nearby chemical environment. Chemiresistors are a class of chemical sensors that rely on the direct chemical interaction between the sensing material and the analyte. The sensing material and the analyte (e.g., a VOC) can interact by covalent bonding, hydrogen bonding, or molecular recognition. Several different materials have chemiresistor properties such as for example metal-oxide semiconductors, some conductive polymers, and nanomaterials like graphene, carbon nanotubes and nanoparticles.

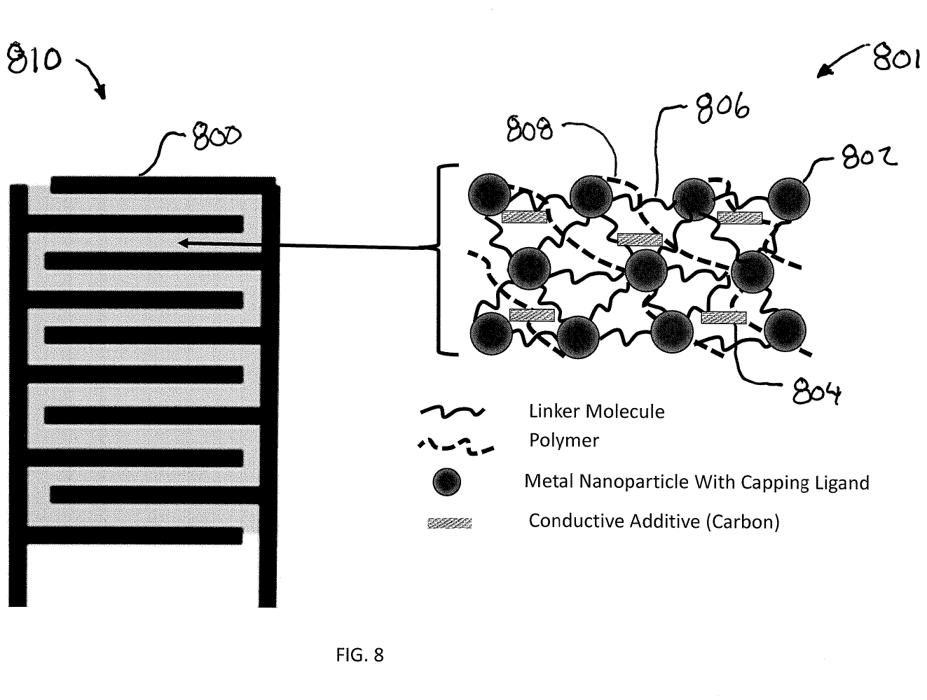

A basic chemiresistor consists of a sensing material that bridges the gap between two electrodes or coats a set of interdigitated electrodes as shown in FIG. 8. The resistance between the electrodes can be measured. The sensing material has an inherent resistance that can be modulated by the presence or absence of the analyte. During exposure, analytes interact with the sensing material. These interactions cause changes in the resistance reading. In some chemiresistors the resistance changes indicate the presence of analyte. In others, the resistance changes are proportional to the amount of analyte present, thus allowing for the amount of analyte present to be measured.

As shown in FIG. 7, in an embodiment multichannel chemiresistor array sensor 112 has eight sensor channels 702a through 702n enclosed in an air sample chamber 704. The sensors of these sensing channels are formed using the following sensing films: MUA-Au2nm, MUA-Au5nm, BDT-Au2nm, BDT-Au5nm, PDT-Au2nm, HDT-Au2nm, NDT-Au2nm, and ND-Au5nm. See Chemiresistive Sensor Array with Nanostructured Interfaces for Detection of Human Breaths with Simulated Lung Cancer Breath VOCs, Guojun Shang, Dong Dinh, Tara Mercer, Shan Yan, Shan Wang, Behnaz Malaei, Jin Luo, Susan Lu, and Chuan-Jian Zhong, ACS Sensors 2023, 23, 16042-16050, DOI: 10.1021/acssensors.2c02839, which is incorporated herein by reference in its entirety. More details regarding the sensing films are provided below with reference to FIG. 8. Also included in air sample chamber 704 is a temperature sensor 706 for measuring the temperature of the air or human breath being analyzed, and a humidity sensor 708 for measuring the humidity of the air or human breath being analyzed. A three-way valve 710 is used to introduce the air or breath being sampled into air sample chamber 704. Between air and breath samples, three-way valve 710 is used to introduce purge air into air sample chamber 704.

FIG. 8 illustrates an embodiment of an example chemiresistor sensor 810 for detecting volatile organic compounds according to the present invention. As shown in FIG. 8, chemiresistor sensor 810 includes a plurality of conductors 800 and a sensing film 801. Sensing film 801 includes metal nanoparticles with capping ligands 802, a conductive additive 804 such as for example carbon, linker molecules 806, and a polymer 808.

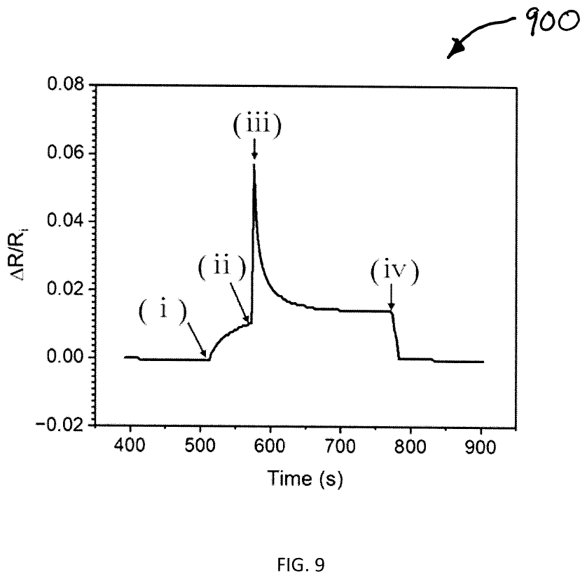

FIG. 9 illustrates an example output 900 of a chemiresistor sensor 810 for detecting volatile organic compounds according to an embodiment of the present invention. As shown in the graph for example output 900, at location (i), purge air is introduced into air sample chamber 704. In embodiments, this purge air is run for about two minutes. At location (ii), a vacuum is drawn in air sample chamber 704 using for example a diaphragm pump (not shown). The pump is run in embodiments for about one minute. At location (iii), the air or human breath to be analyzed is drawn into air sample chamber 704. In the case of human breath, the sample can be introduced into air sample chamber 704 by a releasing valve connecting a sample collection bag 400.

In embodiments, a period of about three minutes is allowed for the air in collection bag 400 to be drawn into air sample chamber 704 and analyzed by the chemiresistor sensor 810 of multichannel chemiresistor array sensor 112. At location (iv), purge air is again introduced for example for about two minutes. In other embodiments, times other than those presented here are used.

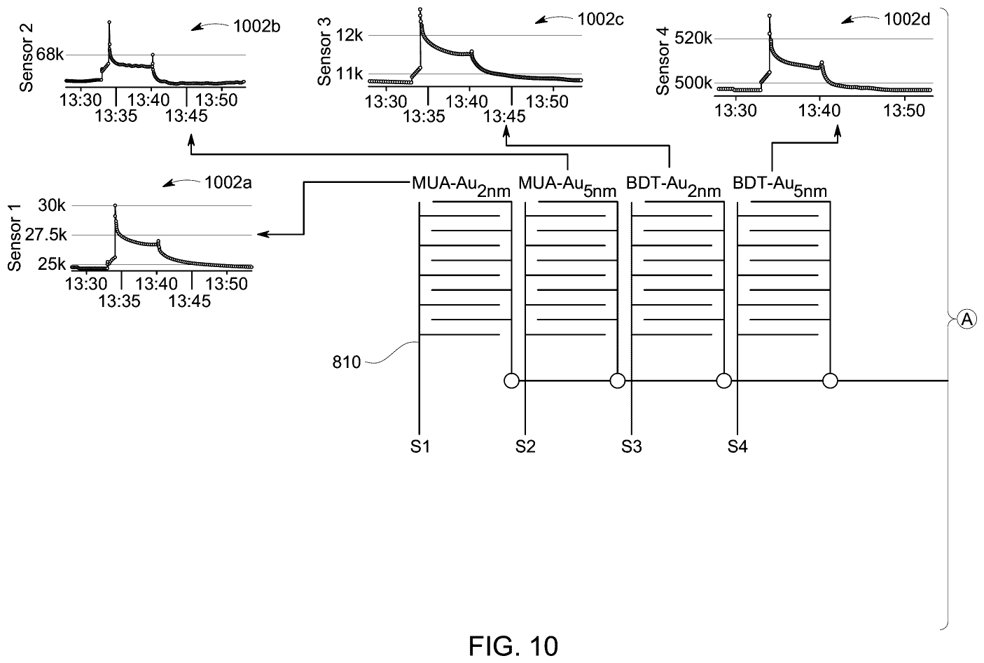

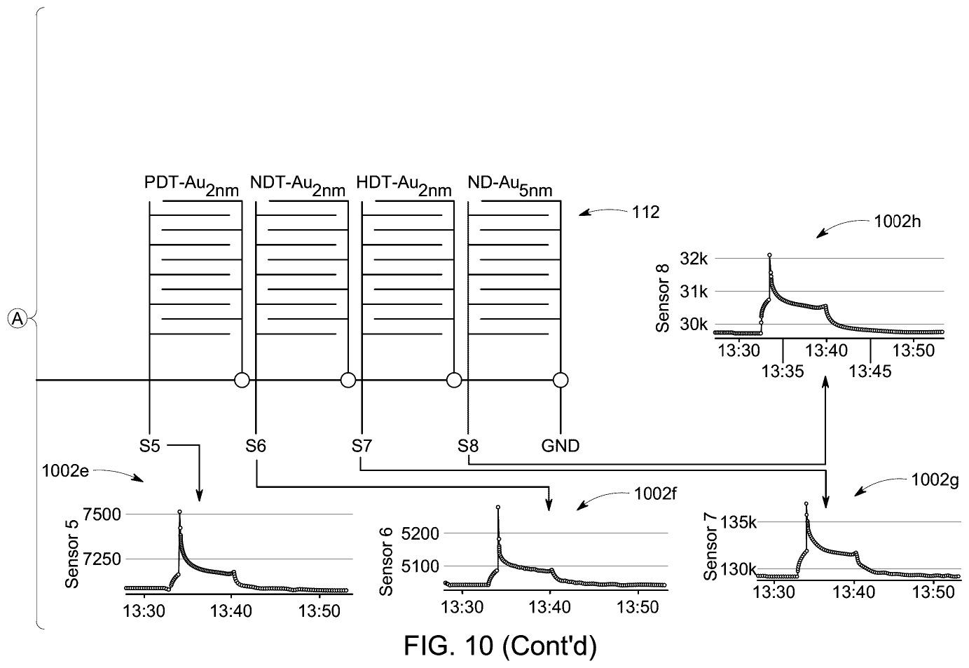

FIG. 10 further illustrates example multichannel chemiresistor array sensor 112 for detecting volatile organic compounds according to an embodiment of the present invention. As shown in FIG. 10, each of the chemiresistor sensor 810 generates its own individual output 1002a though 1002h. The outputs shown represent changes in resistance caused by the presents of VOCs in the air sample being analyzed. As seen in the output signals 900, the response of each chemiresistor sensor 810 is different. In embodiments, the resistance of the different chemiresistor sensors 810 can vary widely. For example, between 30 ohms and 30 megaohms depending on the sensing films used. To accommodate this wide variation in resistance, the sensing channels 602 or multichannel sensor array interface board of the air sampling sensor 112 automatically adjusts it ability to measure resistance to a particular resistance measurement range similar to an auto-ranging multimeter.

FIG. 11 illustrates various statistical methods used to process sensor data according to embodiments of the present invention. As shown in FIG. 11, several different statistical methods can be used for processing the data such as the KNN Classifier method shown in graph 1002, the ANN Classifier method shown in graph 1104, the SVM Classifier method shown in graph 1106, the XGBoost Classifier method shown in graph 1108, and the RF Classifier method shown in graph 1110.

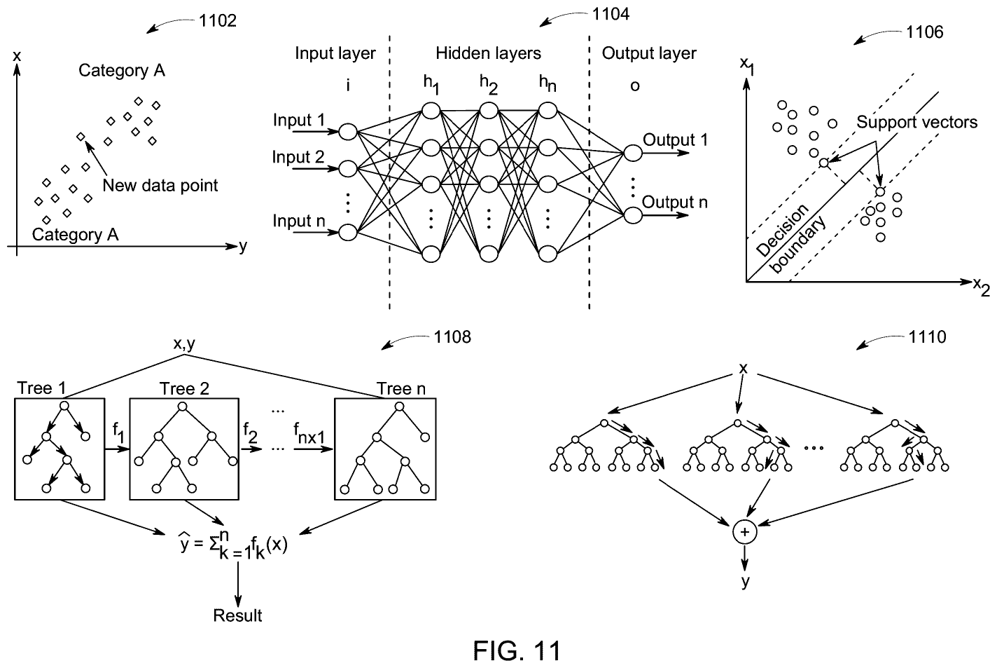

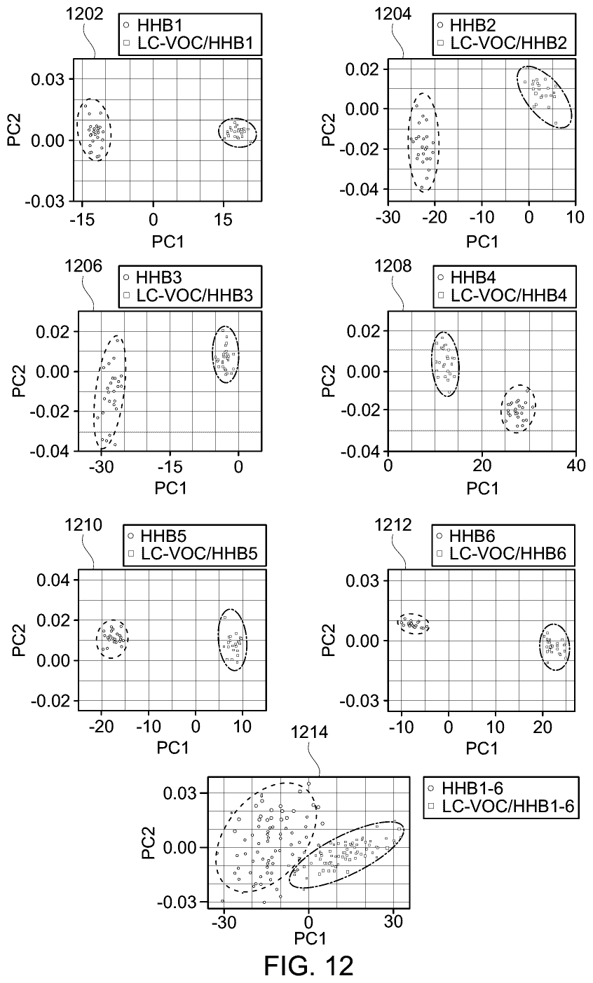

FIG. 12 illustrates various principal component analysis plots of examplary multichannel chemiresistor array sensor 112 for detecting volatile organic compounds according to an embodiment of the present invention. The plots shown in FIG. 12 are principal component analysis plots of sensor data for human breath samples with and without lung cancer specific VOCs such as toluene, 2-butanone, and 2,3,4-trimethyl-pentane, each at 9 ppm. (As indicated in the figures, healthy human breath is represented by HHB, and human breath with lung cancer (LC) specific VOCs is represented by LC-VOC/HHB.) The plots 1202, 1204, 1206, 1208, 1210, and 1212 are for different individuals. Plot 1214 is a combined plot of the six other plots of human breath samples with and without lung cancer specific VOCs shown in FIG. 12. See Chemiresistive Sensor Array with Nanostructured Interfaces for Detection of Human Breaths with Simulated Lung Cancer Breath VOCs, Guojun Shang, Dong Dinh, Tara Mercer, Shan Yan, Shan Wang, Behnaz Malaei, Jin Luo, Susan Lu, and Chuan-Jian Zhong, ACS Sensors 2023, 8, 3, 1328-1338, DOI: 10.1021/acssensors.2c02839, which is incorporated herein by reference in its entirety.

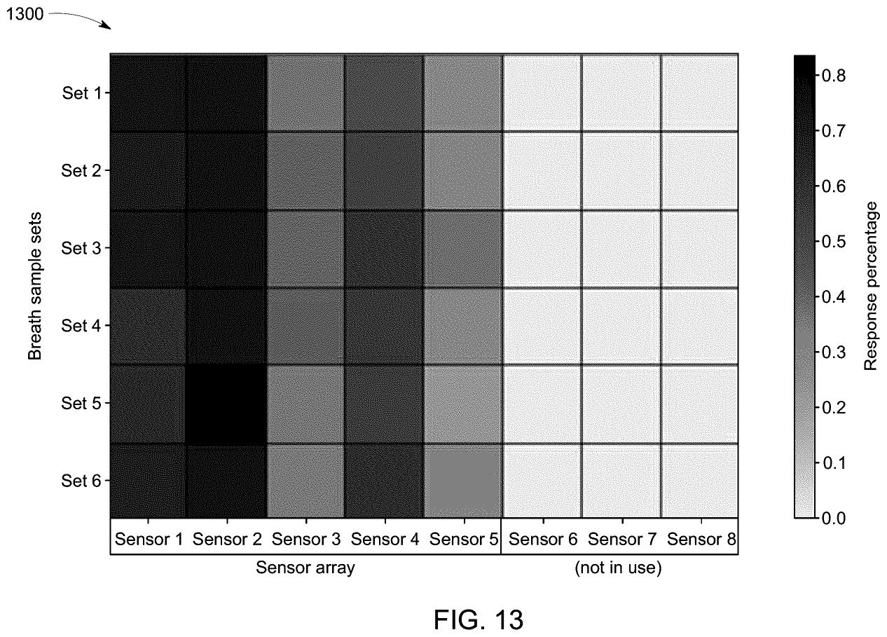

FIG. 13 illustrates a graph 1300 of example sensor responses of a multichannel chemiresistor array sensor to six sets of breath samples according to an embodiment of the present invention. In FIG. 13, the darker squares indicate a larger resistance response change, ((RFinal−RInitial)/RInitial), by a sensor than a lighter square represents. Sensors 6, 7, and 8, were not in use as noted in graph 1300 in order to show a baseline to which the changes in the other sensors can be compared. It should be appreciated that 1-n samples can be tested depending upon the configuration of the array. See, e.g., D. Dinh, G. Shang, S. Yan, J. Luo, A. Huang, L. Yang, S. Lu, C.J. Zhong, A Wireless Sensor Array System Coupled With Al-Driven Data Analysis Towards Remote Monitoring of Human Breaths, IEEE Sensors Journal, 2023, 23,16042-16050.

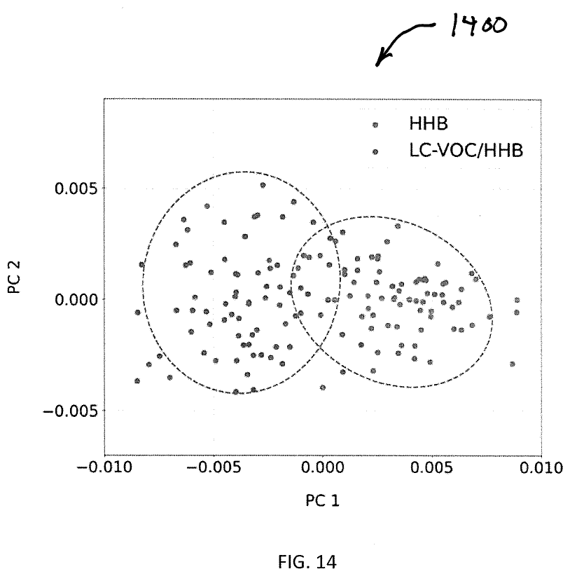

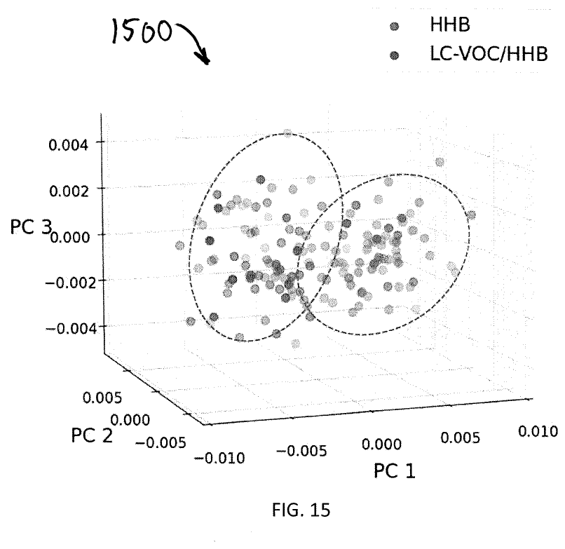

FIGS. 14 and 15 illustrate various principal component analysis plots for the output data of example multichannel chemiresistor array sensor 112 for detecting volatile organic compounds according to an embodiment of the present invention. FIG. 14 depicts a two-dimensional (2D) principal component analysis plot 1400 for the analyzed results of human breath samples with and without spiked lung cancer specific VOCs. FIG. 15 depicts a three-dimensional (3D) principal component analysis plot 1500 for the analyzed results of human breath samples with and without spiked lung cancer specific VOCs.

As can be seen from the figures, the present invention can include a computer-implemented method for detecting VOCs, in air sample, with the computer being one or more processors, such as computer 300 in FIG. 3, which starts with capturing an air sample, such as a human breath sample 102, which can be done with a flexible hose 206, a collection bag 400, other chamber or structure that concentrates an air sample against the chemiresistive sensor array 112. Then the air sample is directed against a chemiresistive sensor array 112, wherein the chemiresistive sensor array 112 produces an electrical signal (FIG. 6) whenever a volatile organic compound is present in the air sample is presented thereagainst. Upon detecting a volatile organic compound in an air sample, the method then includes transmitting, to a wireless communication module 612, detection data from a micro-processing unit 608 electrically coupled to the chemiresistive sensor array 112, the detection data indicating detection of a volatile organic compound; and transmitting received detection data from the wireless communication module 612 to one or more computer devices in wireless communication therewith.

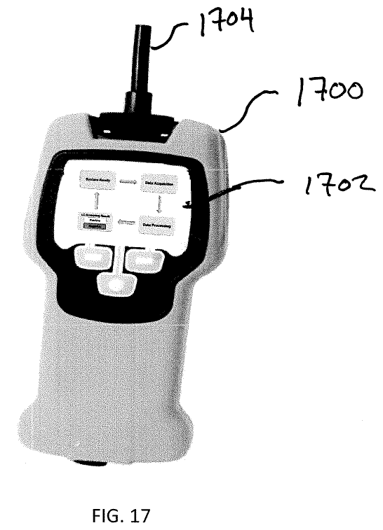

Such computer devices can be cloud-based or other diagnostic computer systems with a wireless connection, such as thought the Internet. The capturing of the air sample can be a human breath sample 102, or alternately, an environmental air sample, such as with handheld air sampling sensor 1700 (FIG. 17).

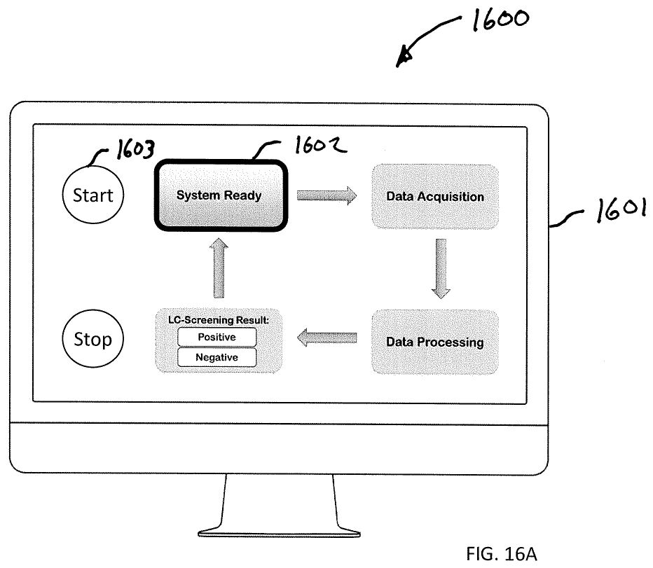

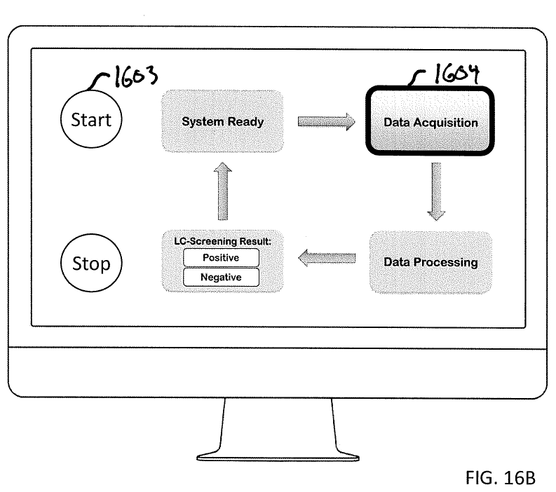

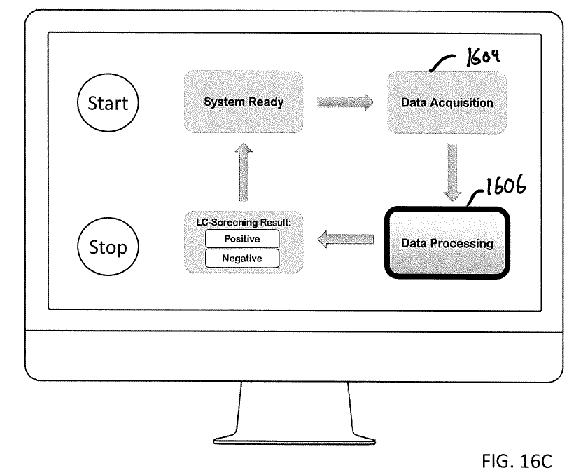

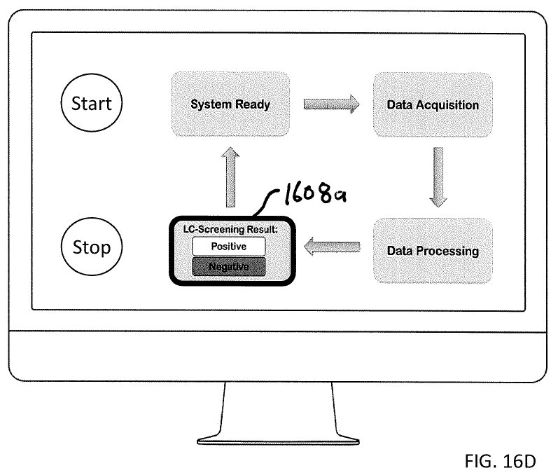

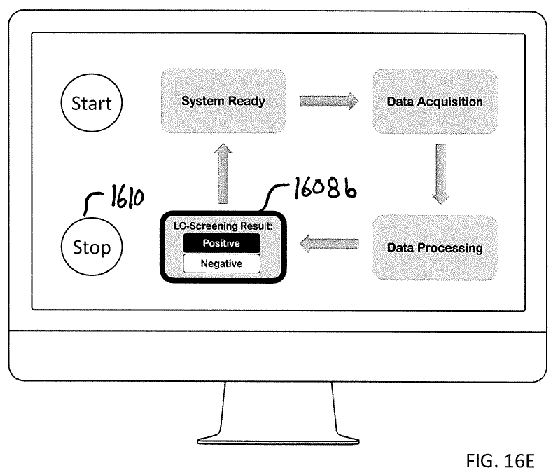

FIGS. 16A-16E illustrate an example graphical user interface (GUI) 1600 for a portable air sampling system 100 for detecting volatile organic compounds according to an embodiment of the present invention. GUI 1600 is displayed on a computer monitor 1601. As shown in FIG. 16A, portable air sampling system 100 starts up and goes to a System Ready state as indicated by graphic 1602 of GUI 1600. To start an air sample analysis, a user clicks on Start button 1603 of GUI 1600. As shown in FIG. 16B, after clicking on Start button 1603, portable air sampling system 100 goes to a “Data Acquisition” state as indicated by graphic 1604 of GUI 1600. In the Data Acquisition state, an air sample is introduced into portable air sampling sensor 220, for example, and the air sample generates causes outputs to be generated by the sensors of multichannel chemiresistor array sensor 112 that are dependent on presence and the concentration of VOCs in the air sample.

As shown in FIG. 16C, after the air sample data has been acquired using multichannel chemiresistor array sensor 112, the state of portable air sampling sensor 220 changes from Data Acquisition as indicated by graphic 1604 to a “Data Processing” state indicated by graphic 1606 of GUI 1600. In the Data Processing state, the output data of the multichannel chemiresistor array sensor 112 is statistically analyzed as described herein to detect the presence or absence of VOCs relevant to lung cancer biomarkers.

After the output data of multichannel chemiresistor array sensor 112 is statistically analyzed, portable air sampling sensor 220 transitions to a “LC-Screening Result” state as shown in FIGS. 16D and 16E. If the LC-Screening Result is Negative, the result is displayed on GUI 1600 as shown in graphic 1608a of FIG. 16D. If the LC-Screening Result is Positive, the result is displayed on GUI 1600 as shown in graphic 1608b of FIG. 16E. After the result is noted or recorded, a user clicks on Stop button 1610, and portable air sampling sensor 220 transitions back to the “System Ready” state as shown in FIG. 16A.

FIG. 17 illustrates an example handheld air sampling sensor 1700 for detecting volatile organic compounds according to an embodiment of the present invention. Handheld air sampling sensor 1700 is used for example to monitor the environment for hazardous VOCs. In embodiments, handheld air sampling sensor 1700 can be used to continuously sample environmental air and indicate an absence of hazardous VOCs. When a hazardous VOC is detected, it is displayed on a screen or other indicator 1702 of handheld air sampling sensor 1700. Air to be analyzed is drawn into handheld air sampling sensor 1700 using a tube 1704.

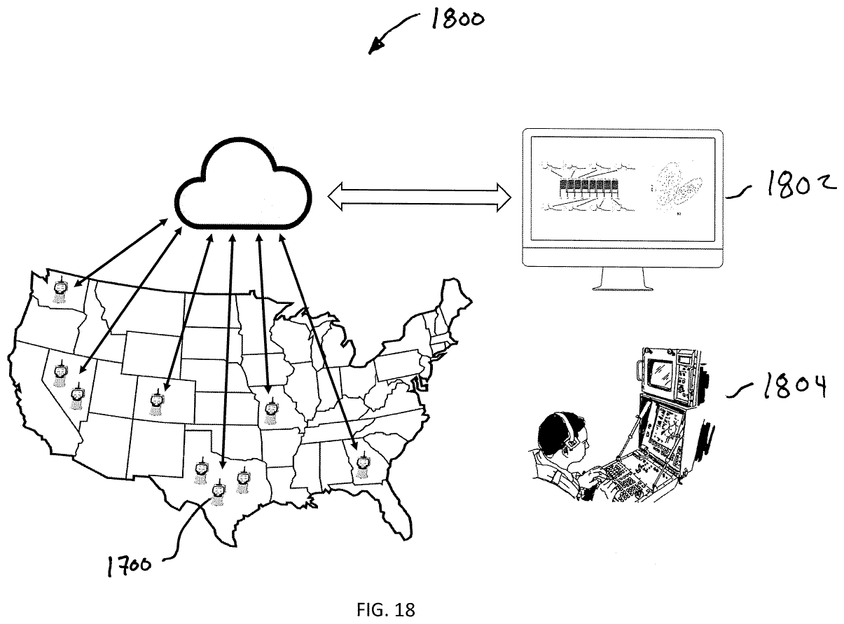

FIG. 18 illustrates an air monitoring system 1800 for remotely monitoring a large number of air sampling sensors such as portable air sampling sensors 1700 for detecting volatile organic compounds according to an embodiment of the present invention. As shown in FIG. 18, the data collected by the portable air sampling sensors 1700 is transmitted for example using the internet or satellites to a central computer or server like server 1802 or computer 1804. The data can then be used to determine the environmental conditions at certain locations and whether hazardous VOCs are present in certain locations.

The corresponding structures, materials, acts, and equivalents of all means or step plus function elements in the claims below, if any, are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of one or more aspects of the invention and the practical application, and to enable others of ordinary skill in the art to understand one or more aspects of the invention for various embodiments with various modifications as are suited to the particular use contemplated.

What is claimed is:

1. An air sampling sensor for detecting volatile organic compounds, comprising:
a chemiresistive sensor array;
a multichannel sensor array interface electronic board with low current electrically coupled to the chemiresistive sensor array;
an analog-to-digital converter electrically coupled to the multichannel sensor array interface board; and
a micro-processing unit electrically coupled to the analog-to-digital converter; wherein the chemiresistive sensor array produces an electrical signal whenever a volatile organic compound is present in an air sample introduced into a chamber housing the chemiresistive sensor array.

2. The air sampling sensor of claim 1, further including a multiplexer sending an output voltage signal from each channel of the multichannel sensor array, the output voltage signal indicative of the sensor resistance, the output voltage signal sent to the analog-to-digital converter.

3. The air sampling sensor of claim 1, wherein the chemiresistive sensor array comprises at least one sensing film from the group comprised of: MUA-Au₂ₙₘ, MUA-Au₅ₙₘ, BDT-Au₂ₙₘ, BDT-Au₅ₙₘ, PDT-Au₂ₙₘ, HDT-Au₂ₙₘ, NDT-Au₂ₙₘ, and ND-Au₅ₙₘ.

4. The air sampling sensor of claim 1, wherein the chemiresistive sensor array comprises at least eight sensing films from the group comprised of: MUA-Au₂ₙₘ, MUA-Au₅ₙₘ, BDT-Au₂ₙₘ, BDT-Au₅ₙₘ, PDT-Au₂ₙₘ, HDT-Au₂ₙₘ, NDT-Au₂ₙₘ, and ND-Au₅ₙₘ.

5. The air sampling sensor of claim 1, wherein the micro-processing unit performs principal component analysis of data from electrical signals produced by the chemiresistive sensor array to determine the presence of volatile organic compounds in the air sample.

6. The air sampling sensor of claim 1, wherein the air sample is a sample of human breath.

7. The air sampling sensor of claim 6, wherein the chemiresistive sensor array comprises sensing films selected to detect the presence of at least one of: toluene, styrene, o-xylene, dimethyl ether, 1,3-pentadiene, methyl hydrazine, ethanol, ethyl benzene, and 2-methly-hexane in the air sample.

8. The air sampling sensor of claim 6, wherein the chemiresistive sensor array comprises sensing films selected to detect the presence of at least one of: 2-ethyl-1-hexanol, 2-ethyl-4-methyl-1-pentanol, 2,3,4-trimethyl-pentant, and 4-methyl-octane in the air sample.

9. The air sampling sensor of claim 6, wherein the chemiresistive sensor array comprises sensing films selected to detect the presence of at least one of: hydrazine, 2-butanone, and 2,2,3-trimethyl-hexane in the air sample.

10. The air sampling sensor of claim 6, wherein the chemiresistive sensor array comprises sensing films selected to detect the presence of volatile organic compounds in the air sample to determine a presence of lung cancer.

11. The air sampling sensor of claim 1, wherein the air sample is an environmental air sample.

12. An air sampling system for detecting volatile organic compounds, comprising:
an air sampling sensor that includes:
a chemiresistive sensor array;
a multichannel sensor array interface board electrically coupled to the chemiresistive sensor array; and
a micro-processing unit electrically coupled to the multichannel sensor array,
wherein the chemiresistive sensor array produces an electrical signal
whenever a volatile organic compound is present in an air sample introduced into
a chamber housing the chemiresistive sensor array; and
a wireless communication module that transmits data from the micro-processing unit to a computer.

13. The air sampling system of claim 12, wherein the computer is a cloud server computer.

14. The air sampling system of claim 12, further comprising:
a second air sampling sensor that transmits data from the second air sampling sensor to the computer.

15. The air sampling system of claim 13, wherein the data transmitted to the computer relates to environmental air samples.

16. The air sampling system of claim 13, wherein the data transmitted to the computer relates to human breath samples.

17. The air sampling system of claim 12, further comprising a plurality of chemiresistive sensor arrays configured to test multiple air samples.

18. A computer implemented method of detecting volatile organic compounds within an air sample, comprising:
capturing an air sample;
directing the air sample against a chemiresistive sensor array, wherein the chemiresistive sensor array produces an electrical signal whenever a volatile organic compound is present in an air sample is presented thereagainst;
upon detecting a volatile organic compound in an air sample, transmitting, to a wireless communication module, detection data from a micro-processing unit electrically coupled to the chemiresistive sensor array, the detection data indicating detection of a volatile organic compound; and
transmitting received detection data from the wireless communication module to one or more computer devices in wireless communication therewith.

19. The method of claim 18, wherein capturing the air sample is capturing a human breath sample.

20. The method of claim 18, wherein capturing the air sample is capturing an environmental air sample.
